(19) United States
(12) Patent Application Publication
(10) Pub. No.: US 2021/0106619 A1
(43) Pub. Date: Apr. 15, 2021

(54) NK-92 Bone and Brain targeting

(71) Applicant: NantKwest, Inc., San Diego, CA (US)

(72) Inventor: John H. Lee, San Diego, CA (US)

(21) Appl. No.: 17/024,155

(22) Filed: Sep. 17, 2020

Related U.S. Application Data
(60) Provisional application No. 62/913,644, filed on Oct. 10, 2019.

(51) Int. Cl. A61K 35/17; A61K 49/00; C07K 14/725; C07K 14/705

(52) U.S. Cl. CPC A61K 35/17; A61K 49/005; C07K 14/7051; C07K 14/70517; C07K 2317/622; C07K 2317/53; C07K 2319/02; C07K 2319/03; C07K 14/70521

(57) ABSTRACT

Compositions of luminescence labeled activated natural killer (aNK) cells are utilized in methods of in vivo bioluminescence imaging (BLI) for assaying and identifying genetically modified NK cells capable of targeting selected anatomical locations (e.g., bone and/or brain) or targeting selected diseased cells at selected sites.

This application claims priority to our copending US Provisional patent application with the Ser. No. 62/913,644, filed Oct. 10, 2019, incorporated by reference herein.

### SEQUENCE LISTING

The content of the ASCII text file of the sequence listing named 104077.0015PCT_ST25.txt, which is 13 KB in size was created on Sep. 4, 2020 and electronically submitted via EFS-Web along with the present application, and is incorporated by reference in its entirety.

### FIELD OF THE INVENTION

The present disclosure relates to compositions and methods for luminescence labeling of natural killer (NK) cells for administration to a subject for in vivo imaging and localization, and especially relates to the localization of engineered NK cells.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Specific and effective targeting of cellular therapies for the treatment of disease is a focus of many research studies in view of the nuances and considerations specific to a disease class as well and each specific disease indication. For the treatment of cancers which have metastasized to various locations in a patient's body, the penultimate goal is to target therapies which are specific to the cancer cells and that able to reach most if not all of the anatomical locations harboring the cancer cells. To that end, cells can be genetically modified to express targeting molecules. However, it is not always evident that such targeting will effectively direct the therapeutic cells to the desired location.

Most of the traditional methods of localizing and quantifying potential targeting therapies rely on sacrificing an animal that has received the treatment, followed by analysis of the animal's various tissues and organs. While these ex vivo methods have indeed provided valuable information to advance the field of targeting therapeutics, a large number of animals is required for a given study. Resources aside, these ex vivo methods often have limited sensitivity with respect to the localization of a relatively small number of cells, which if detected at all, could be identified in a given tissue sample as an artifact of the study protocol.

To improve localization information, in vivo bioluminescence (BLI) can be used in some cases. BLI is an optical molecular imaging technique used to visualize molecular and cellular processes to follow the fate of cells in real time with high sensitivity using luciferase-based gene reporters. Specific luciferase-based studies using BLI have monitored labeled tumor cells in vivo in mice to visualize tumor progression (Sweeney et al., 1999, PNAS, 96:12044-12049) and metastatic formation (Minn et al., 2005, J. Clin. Inv., 115:44-55).

For cancer immunotherapies, adoptively transferred tumor-specific cytotoxic lymphocytes hold promise for the treatment of patients with tumor malignancies. Natural killer (NK) cells are cytotoxic lymphocytes that constitute a major component of the innate immune system. Natural killer (NK) cells, generally representing about 10-15% of circulating lymphocytes, bind and kill targeted cells, including virus-infected cells and many malignant cells, non-specifically with regard to antigen and without prior immune sensitization. (Herberman et al., 1981, Science 214:24.) The NK-92 cell line is a cytolytic cancer cell line which was discovered in the blood of a subject suffering from a non-Hodgkin's lymphoma and then immortalized ex vivo. NK-92 cells are derived from NK cells, but lack the major inhibitory receptors that are displayed by normal NK cells, while retaining the majority of the activating receptors. NK-92 cells do not, however, attack normal cells nor do they elicit an unacceptable immune rejection response in humans.

While the capabilities and potential of NK-92 cells including modified NK-92 cells are an advancement for tumor malignancies, the therapeutic readout is typically gross tumor growth (e.g., regression), leaving the full penetrance of these NK cells unknown. Furthermore, while tumor regression may occur with administration of some therapies, metastasis is not necessarily evaded, and in some cases, it is merely postponed until relapse.

Accordingly, having the capability to target cellular therapies (e.g., NK-92 cells) to locations of interest, as well as develop cellular therapies with increased penetrance to more or all of a patient's cancer cells, is an unmet need in cancer therapies.

### SUMMARY OF THE INVENTION

The inventors have discovered compositions and methods that enable the in vivo bioluminescence imaging (BLI) of luminescence labeled activated natural killer (aNK) cells in a subject when the labeled activated aNK cells are administered to the subject. Typically, the aNK cells are genetically modified NK-92 cells expressing a luciferase protein. More typically, the genetically modified NK-92 cells constitutively express a firefly luciferase protein.

Unexpectedly, the inventors discovered that the genetically modified NK-92 cells expressing a luciferase protein preferentially located to specific tissues and appeared to home to target cells located in the lungs, and to a lesser degree to the bone, heart, spleen, kidneys, brain, and liver. Therefore, in preferred aspects the method includes identifying luminescence labeled genetically modified NK-92 cells capable of homing to corresponding target cells located in the lungs (or other target organ such as brain and/or bone) of the subject.

In another aspect of the inventive subject matter, contemplated methods also include in vivo bioluminescence imaging for identifying a luminescence labeled genetically modified NK-92 cells in tissues that were targeted by the genetically modified NK-92 cells via a recombinantly expressed homing receptor, CAR, and/or other targeting molecule. Advantageously, such methods allow for confirmation of appropriate targeting of NK cells, even where such NK cells would otherwise locate or home to a different tissue (e.g., lungs). Typically, the targeted tissue will include diseased cells such as cancer cells and/or virus-infected cells.

While in some aspects the contemplated in vivo BLI methods include luminescence labeled modified NK-92 cells capable of homing or preferentially targeting diseased cells, other aspects include luminescence labeled unmodified NK-92 cells that are not specifically targeted to a diseased cell, but preferentially target an anatomical location (e.g., brain or bone).

In one aspect of the inventive subject matter, the inventors contemplate a method of assaying in vivo localization of luminescence labeled activated natural killer (aNK) cells in a subject, wherein such methods includes a step of administering a dose of luminescence labeled aNK cells to the subject and a further step of imaging the subject with bioluminescence imaging (BLI). Most typically, the luminescence labeled aNK cells is transfected with a recombinant nucleic acid expressing a luciferase protein, the luciferase protein is a firefly luciferase protein, and/or the luciferase protein is constitutively expressed.

In some embodiments, the luminescence labeled aNK cells are unmodified or modified NK-92 cells (e.g., NK-92-CD16, NK-92-CD16-γ, NK-92-CD16-ζ, NK-92-CD16(F176V), NK-92MI, or NK-92CI). In other embodiments, the luminescence labeled aNK cells are modified NK-92 cells transfected with a membrane bound recombinantly expressed chimeric antigen receptor (CAR) that comprises in a single polypeptide chain an extracellular binding domain, a hinge domain, a transmembrane domain, and a FcεRIγ signaling domain. Where desired, the modified NK cells may further comprise a recombinantly expressed cytokine (IL-2 or IL-15, each optionally containing an endoplasmic retention sequence) and/or a recombinantly expressed CD16. Where the NK-92 cells express a CAR, the extracellular binding domain typically comprises a scFv that may, for example, specifically bind to a tumor-specific antigen, a tumor associated antigen, or a patient- and tumor-specific antigen. Exemplary tumor-specific antigens include NKG2D ligands, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAP, WT-1, PSMA, NY-ESO1, AFP, CEA, CTAG1B, CD33, CD19, CD20, HER-2, CD123, PD-L1, IGF1R, CSPG4, BCMA, and B7-H4. Alternatively, the extracellular binding domain may specifically bind to a virus-specific antigen. In such methods, it is contemplated that the dose of luminescence labeled aNK cells comprises 1.0×10⁶ to 1.0×10⁹ cells.

In another aspect of the inventive subject matter, the inventors contemplate a method of identifying a luminescence labeled activated natural killer (aNK) cells capable of targeting a selected anatomical location in a subject, and such method will typically comprise step of administering a dose of luminescence labeled aNK cells to the subject, and a step of imaging the subject with bioluminescence imaging (BLI) at the selected anatomical location.

Most typically, the luminescence labeled aNK cells is transfected with a recombinant nucleic acid expressing a luciferase protein. Among other suitable choices, the luciferase protein is a constitutively expressed firefly luciferase protein, and the selected anatomical location is selected from lungs, heart, spleen, kidneys, liver, bone, and/or brain. In further aspects, the luminescence labeled aNK cells are unmodified or modified NK-92 cells as noted above.

Therefore, the inventors also contemplate a composition for in vivo bioluminescence imaging that comprises a modified NK-92 cell genetically modified to produce luminescence, and to recombinantly express an Fc receptor, a chimeric antigen receptor (CAR), a cytokine, or a homing receptor. For example, the NK-92 cell may be genetically modified to recombinantly express a luciferase protein, and/or the luciferase protein is a constitutively expressed firefly luciferase protein. With respect to the modified NK-92 cells, the same considerations as noted above apply.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1A depicts exemplary results of in vivo imaging of SCID mice injected with firefly luciferase-labeled NK-92 cells and imaged after lateral tail vein injection at 0 (approximately 20 minutes), 2, 4, 6, 8, and 18 hours after injection, as indicated, according to embodiments of the present invention.

FIG. 1B is a graph of the total radiance in the lung in vivo of all 3 mice in FIG. 1A over the measured time points on a log scale, as indicated, according to embodiments of the present invention.

FIG. 1C is a graph of the total radiance in the lung in vivo of all 3 mice in FIG. 1A over the measured time points on a linear scale, as indicated, according to embodiments of the present invention.

FIG. 2A depicts exemplary results of ex vivo imaging of the heart, lungs, liver, spleen, kidneys, femurs, and brain extracted from the labeled mice in FIG. 1A, according to embodiments of the present invention.

FIG. 2B is a graph of the total radiance in the lung ex vivo of all 3 mice in FIG. 2A over the measured time points on a log scale, as indicated, according to embodiments of the present invention.

FIG. 3A depicts exemplary results of in vivo imaging of NSG™ and nude mice injected by lateral tail vein injection with firefly luciferase-labeled NK-92 cells twice weekly for 4 consecutive weeks, followed by imaging at 0 (approximately 20 minutes), 2, and 4 hours after the last injection, according to embodiments of the present invention.

FIG. 3B is a graph of the total radiance in vivo in the thorax in the NSG mice (circles) and nude mice (squares) in FIG. 3A over the measured time points on a log scale, according to embodiments of the present invention.

FIG. 4 depicts exemplary results of ex vivo imaging of the heart, lungs, liver, spleen, kidneys, femurs, and brain extracted from the labeled NSG mice and nude mice of FIG. 3A, according to embodiments of the present invention.

FIG. 5 depicts an exemplary luciferase encoding vector according to the inventive subject matter.

### DETAILED DESCRIPTION

The inventors have now discovered a method of in vivo bioluminescent imaging (BLI) utilizing compositions comprising luminescence labeled natural killer (e.g., NK-92) cells for ascertaining localization, targeting, and/or homing of genetically engineered natural killer cells to specific targeted diseased cells/tissues and/or to preferential anatomical locations harboring the diseased cells. As used herein the term “luminescence labeled cells” refers to cells that have been modified (e.g., genetically modified via transfection) to be capable of generating a luminescence signal from a luminescent substrate.

In an effort to ascertain the anatomical location of previously administered NK-92 cells, the inventors have used in vivo and ex vivo BLI. Unexpectedly, and as disclosed in more detail herein, luminescence labeled NK-92 cells predominantly localized to the lungs relative to a large number of other tissues. Notably, the luminescence labeled NK-92 cells did not have any recombinant nucleic acid that encoded a targeting molecule that would target lung tissue. Accordingly, contemplated methods include NK cell therapy directed towards the lungs via administration of luminescence labeled or unmodified NK-92 cells.

Furthermore, using the in vivo bioluminescent imaging methods as disclosed herein, the inventors also contemplate methods for identifying luminescence labeled activated natural killer (aNK) cells capable of targeting a selected anatomical location(s). That is, using luminescence labeled NK-92 cells that have been modified to express various homing and/or targeting receptors, it is now possible to monitor or verify if the intended targeting for the modified NK cells will indeed direct the NK cell to the desired target. For example, the penetrance of these cytotoxic cells may be altered to common sites associated with specific cancers or infections, for example, using cancer specific receptors or CARs.

Aspects of the inventive subject matter with respect to in vivo imaging include labeling the engineered NK-92 cells with luminescence. Any suitable method for labeling the cells may be used following established methods known in the art. That is any luminescence label or recombinant protein capable of producing luminescence or radio labeling may be introduced into the suitable NK cells and subsequently be imaged in vivo real time and/or ex vivo.

In typical embodiments, the luminescence labeling of NK-92 cells includes the introduction of one or more recombinant gene or genes that express a protein product capable of producing luminescence from an appropriate substrate. As will be readily appreciated, the recombinant gene(s) may be transfected or transduced into the NK-92 cells to express the relevant protein prior to and/or after in vivo administration (e.g., injection). It should be recognized that suitable recombinant gene(s) may be transfected into NK cells as recombinant DNA or RNA. Additionally, it should be noted that expression may be inducible or constitutive, depending on the desired timing of localization and protocol.

In more typical embodiments, the luminescence labeling of NK-92 cells includes expression of one or more recombinant luciferase enzymes. The enzymatic interaction of luciferase with its substrate luciferin generates light. In preferred embodiments, a recombinant luciferase gene is transfected into the cells for inducible or constitutive expression. Luciferin may be co-transfected with the luciferase, or the luciferin substrate may be provided separately. In order to determine the anatomical localization of so modified NK-92 cells regardless of the molecular environment at a location (e.g., an organ such as the brain or bone), constitutive expression of luciferin together with luciferase is preferably used. In preferred embodiments, a recombinant firefly luciferase gene is transfected into NK-92 cells and expressed constitutively. The transfected cells may be exposed to or loaded with luciferin or a luciferin analog after transfection (e.g., before administration, or after administration of the NK cells). Established luciferase methodologies utilizing luciferin and luciferin analogs for in vivo imaging are described in the art. Non-limiting examples of naturally secreted luciferases include GLuc (Gaussia luciferase) and Renilla luciferase. Constructs for luciferase expression together with the expression or addition of luciferin or luciferin analogs are described in Tannous et al., 2005, Mol Ther, 11:435-443; Wurdinger et al., 2008, Nature Methods, 5:171-173, Wehrman et al., 2006, Nature Methods, 3:295-301, and Fan et al., 2007, Assay Drug Dev Technol, 5: 127-136.

In typical embodiments, modification of the luminescence labeled NK-92 cells includes the introduction (e.g., by cell transfection) of a nucleic acid construct for expressing a recombinant homing receptor and/or a chimeric antigen receptor (CAR) as disclosed herein. Genetic modification of the NK-92 cells as contemplated may be performed in numerous manners, and all known manners are deemed suitable for use hereon. Moreover, it should be recognized that NK cells can be transfected with DNA or RNA, and the particular choice of transfection will at least in part depend on the type of desired recombinant cell and transfection efficiency. For example, where it is desired that NK cells are stably transfected, linearized DNA may be introduced into the cells for integration into the genome. On the other hand, where transient transfection is desired, circular DNA or linear RNA (e.g., mRNA with polyA+ tail) may be used.

The term “NK-92” refers to natural killer cells derived from the highly potent unique cell line described in Gong et al. (Leukemia 1994 April; 8(4):652-8), and which are commercially available (NantKwest, Inc (9920 Jefferson Blvd., Culver City, Calif. 90232) as aNK cells. Thus, the terms “aNK cell” and “NK-92 cell” are used interchangeably herein. The immortal NK cell line was originally obtained from a patient having non-Hodgkin's lymphoma. Unless indicated otherwise, the term “NK-92” is intended to refer to the cells contained in the original NK-92 cell lines (and progeny thereof) as well as NK-92 cell lines that have been modified (e.g., by introduction of exogenous genes). NK-92 cells and exemplary and non-limiting modifications thereof are described in U.S. Pat. Nos. 7,618,817; 8,034,332; 8,313,943; 9,181,322; 9,150,636; and published U.S. application Ser. Nos. 10/008,955, 16/529,029, and PCT Application No. PCT/US19/33407 all of which are incorporated herein by reference in their entireties, and include wild type NK-92, NK-92-CD16, NK-92-CD16-γ, NK-92-CD16-ζ, NK-92-CD16(F176V), NK-92MI, and NK-92CI. NK-92 cells are known to persons of ordinary skill in the art, to whom such cells are readily available from NantKwest, Inc.

The term “haNK” refers to natural killer cells derived from aNK cells, modified to express CD16 on the cell surface (hereafter, “CD16+ NK-92 cells” or “haNK cells”). In some embodiments, the CD16+ NK-92 cells comprise a high affinity CD16 receptor on the cell surface. The term “taNK” refers to natural killer cells derived from aNK cells, modified to express a chimeric antigen receptor (hereafter, “CAR-modified NK-92 cells” or “taNK cells”). The term “t-haNK” refers to natural killer cells derived from aNK cells, modified to express CD 16 on the cell surface and to express a chimeric antigen receptor (hereafter, “CAR-modified CD16+ NK-92 cells” or “t-haNK cells”). In some embodiments, the t-haNK cells express a high affinity CD16 receptor on the cell surface.

A “modified NK-92 cell” refers to an NK-92 cell that expresses an exogenous gene or protein, such as an Fc receptor, a CAR, a cytokine (such as IL-2 or IL-15), a homing receptor, and/or a suicide gene. In some embodiments, the modified NK-92 cell comprises a vector that encodes for a transgene, such as an Fc receptor, a CAR, a cytokine (such as IL-2 or IL-15), homing receptor, and/or a suicide gene. In one embodiment, with the exclusion of or in addition to any recombinant genes for luminescence, the modified NK-92 cell expresses at least one transgenic protein.

In another aspect of the inventive subject matter, the genetically engineered luminescence labeled NK-92 cell may be modified to express the high-affinity Fcγ receptor (CD16). Sequences for high-affinity variants of the Fcγ receptor are well known in the art (see e.g., Blood 2009 113:3716-3725), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells. Such cells may then be additionally genetically modified to a CAR as further described in more detail below.

Genetic modification of the luminescence labeled NK-92 cells contemplated herein can be performed in numerous manners, and all known manners are deemed suitable for use herein. Moreover, it should be recognized that NK cells can be transfected with DNA or RNA, and the particular choice of transfection will at least in part depend on the type of desired recombinant cell and transfection efficiency. For example, where it is desired that NK cells are stably transfected, linearized DNA may be introduced into the cells for integration into the genome. On the other hand, where transient transfection is desired, circular DNA or linear RNA (e.g., mRNA with polyA+ tail) may be used.

For example, it is contemplated that the luminescence labeled NK-92 cells is transfected with a recombinant nucleic acid including a segment that encodes a CAR that includes FcεRIγ signaling domain, and preferably also a segment that encodes a cytokine to provide autocrine growth stimulation (e.g., IL-2, IL-2 that is modified with an ER retention sequence, IL-15, or IL-15 that is modified with an ER retention sequence) and/or a segment that encodes a CD16 or high affinity CD16^{158V}. As will be readily appreciated, inclusion of a cytokine that provides autocrine growth stimulation will render the modified recombinant independent of exogenous cytokine addition, which will render large scale production of such cells economically feasible. Likewise, where the modified recombinant also expresses CD16 or a high affinity CD16^{158V}, such cells will have further enhanced ADCC characteristics and with that further improved targeted cytotoxicity.

Of course, it should be recognized that the recombinant nucleic acid that encodes that cytokine and/or the CD16 or high affinity CD16^{158V} can be integrated in to the genome of the luminescence labeled NK-92 cell, or can be supplied as an extrachromosomal unit (which may be a linear or circular DNA, or a linear RNA, virally delivered or via chemical, mechanical, or electrical transfection). For example, recombinant NK-92 cells expressing IL-2ER and CD16158V are known as haNK cells (Oncotarget 2016 Dec. 27; 7(52): 86359-86373) and can be transfected with a recombinant nucleic acid that includes a segment that encodes a CAR that includes FcεRIγ signaling domain. Once more, such recombinant nucleic acid may comprise further segments that may encode additional immunotherapeutic proteins, such as N-803, TxM-type compounds, IL-8 traps, TGF-β traps, etc. Likewise, NK-92 cells may already be transfected with a cDNA that encodes IL-2 (e.g., NK-92MI, ATCC CRL-2408). Such cells can then be further transfected with a recombinant nucleic acid that includes a segment that encodes a CAR that includes FcεRIγ signaling domain along with a segment that encodes a CD16 or high affinity CD16^{158V}.

On the other hand, luminescence labeled NK-92 cells may also be transfected with a recombinant nucleic acid that includes a segment that encodes a CAR with a FcεRIγ signaling domain, a segment that encodes a cytokine to provide autocrine growth stimulation (e.g., IL-2, IL-2 that is modified with an ER retention sequence, IL-15, or IL-15 that is modified with an ER retention sequence) and a segment that encodes a CD16 (SEQ ID NO:1 or high affinity CD16^{158V} (SEQ ID NO:2, encoded by SEQ ID NO:3). Most typically, such recombinant nucleic acid will be arranged as a tricistronic construct. As noted before, such constructed can be an extrachromosomal circular plasmid, a linear DNA (which may be integrated into the genome of the NK cell), or a linear RNA. Such nucleic acids will typically be transfected into the cells in a manner well known in the art (e.g., electroporation, lipofection, pressure assisted transfection, ballistic gene transfer, etc.). Similarly, the nucleic acid may be delivered to the cell via a recombinant virus. Therefore, NK cells suitable for use herein include NK-92 cells (which may be transfected with a tricistronic construct encoding a CAR, a CD16 or variant thereof, and a cytokine or variant thereof), a genetically modified NK cell or NK-92 cell that expresses a CD16 or variant thereof or a cytokine or variant thereof (which may be transfected with a nucleic acid encoding a CAR and a CD16 or variant thereof or a cytokine or variant thereof), and a genetically modified NK cell or NK-92 cell that expresses a CD16 or variant thereof and a cytokine or variant thereof (which may be transfected with a nucleic acid encoding a CAR).

In preferred embodiments, it should therefore be noted that the genetically modified NK cell (especially where the cell expresses a CAR and CD16 or variant thereof) will exhibit three distinct modes of cell killing: General cytotoxicity which is mediated by activating receptors (e.g., an NKG2D receptor), ADCC which is mediated by antibodies bound to a target cell, and CAR mediated cytotoxicity.

Consequently, it should be appreciated that the manner of transfection will at least in part depend on the type of nucleic acid employed. Therefore, viral transfection, chemical transfection, mechanical transfection methods are all deemed suitable for use herein. For example, in one embodiment, the vectors described herein are transient expression vectors. Exogenous transgenes introduced using such vectors are not integrated in the nuclear genome of the cell; therefore, in the absence of vector replication, the foreign transgenes will be degraded or diluted over time.

In another embodiment, the vectors described herein allow for stable transfection of cells. In one embodiment, the vector allows incorporation of the transgene(s) into the genome of the cell. Preferably, such vectors have a positive selection marker and suitable positive selection markers include any genes that allow the cell to grow under conditions that would kill a cell not expressing the gene. Non-limiting examples include antibiotic resistance, e.g. geneticin (Neo gene from Tn5).

As noted earlier with respect to transfection of contemplated NK cells, cells may be transfected with a vector (e.g., plasmid vector). In one embodiment, the vector is a viral vector. As would be understood by one of skill in the art, any suitable vector can be used, and suitable vectors are well-known in the art. Moreover, vectors may be transfected as linear DNA or as circular DNA.

In still other embodiments, the cells are transfected with mRNA encoding the protein of interest (e.g., the CAR). Transfection of mRNA results in transient expression of the protein. In one embodiment, transfection of mRNA into luminescence labeled NK-92 cells is performed immediately prior to administration of the cells. In one embodiment, “immediately prior” to administration of the cells refers to between about 15 minutes and about 48 hours prior to administration. Preferably, mRNA transfection is performed about 5 hours to about 24 hours prior to administration. In at least some embodiments as described in more detail below, NK-92 cell transfection with mRNA resulted in unexpectedly consistent and strong expression of the CAR at a high faction of transfected cells. Moreover, such transfected cells also exhibited a high specific cytotoxicity at comparably low effector to target cell ratios.

With respect to contemplated CARs it is noted that the NK-92 cells will be genetically modified to express the CAR as a membrane bound protein exposing a portion of the CAR on the cell surface while maintaining the signaling domain in the intracellular space. Most typically, the CAR will include at least the following elements (in order): an extracellular binding domain, a hinge domain, a transmembrane domain, and an FcεRIγ signaling domain.

In preferred embodiments, the cytoplasmic domain of the CAR comprises or consists of a signaling domain of FcεRIγ. Notably, and as described in more detail below, the FcεRIγ signaling domain provide for substantially increased expression levels of the CAR as much as for significantly extended cytotoxicity over time. For example, the FcεRIγ signaling domain comprises or consists of or consists essentially of the amino acid sequence of SEQ ID NO:4. In some embodiments, the FcεRIγ cytoplasmic domain is the sole signaling domain. However, it should be appreciated that additional elements may also be included, such as other signaling domains (e.g., CD28 signaling domain, CD3 signaling domain, 4-1BB signaling domain, etc.). These additional signaling domains may be positioned downstream of the FcεRIγ cytoplasmic domain and/or upstream of the FcεRIγ cytoplasmic domain.

In some embodiments, the FcεRIγ signaling domain comprises or consists of or consists essentially of an amino acid sequence having at least about 85%, 90%, 95%, 96%, 97%, 98%, or 99% sequence homology to the amino acid sequence of SEQ ID NO:4. In alternative embodiments, the cytoplasmic domain of the CAR may also comprise a signaling domain of CD3 zeta (CD3ζ). In one embodiment, the cytoplasmic domain of the CAR consists of a signaling domain of CD3 zeta. In one embodiment, the CD3 zeta signaling domain comprises or consists of or consists essentially of the amino acid sequence of SEQ ID NO:5. In some embodiments, the CD3 zeta signaling domain comprises or consists of or consists essentially of an amino acid sequence having at least about 85%, 90%, 95%, 96%, 97%, 98%, or 99% sequence homology to the amino acid sequence of SEQ ID NO:5.

The CAR may comprise any suitable transmembrane domain. In one aspect, the CAR comprises a transmembrane domain of CD28. In one embodiment, the CD28 transmembrane domain comprises or consists of or consists essentially of the amino acid sequence of SEQ ID NO:6 (encoded by nucleic acid with the SEQ ID NO:7). In one embodiment, the CD28 transmembrane domain comprises or consists of or consists essentially of an amino acid sequence having at least about 85%, 90%, 95%, 96%, 97%, 98%, or 99% sequence homology to the amino acid sequence of SEQ ID NO:6. In other embodiments, the transmembrane domain may also be a 4-1BB transmembrane domain.

The CAR may comprise any suitable hinge region. In one aspect, the CAR comprises a hinge region of CD8. In one embodiment, the CD8 hinge region comprises or consists of or consists essentially of the amino acid sequence of SEQ ID NO:8 or SEQ ID NO:9. In one embodiment, the CD8 hinge region comprises or consists of or consists essentially of an amino acid sequence having at least about 85%, 90%, 95%, 96%, 97%, 98%, or 99% sequence homology to the amino acid sequence of SEQ ID NO:8 or SEQ ID NO:9. Such region may be encoded by a nucleic acid having the sequence of SEQ ID NO:10.

Therefore, contemplated CARs will include a general structure of a desired antigen binding domain that is coupled to a hinge domain, which is coupled to a transmembrane domain, which is coupled to a signaling domain. Viewed from another perspective, contemplated CARs may have a desired binding domain (typically as a scFv fragment) that is then coupled to a multifunctional hybrid protein that comprises, consists of, or essentially consists of a hinge domain, which is coupled to a transmembrane domain, which is coupled to a signaling domain. For example, such multifunctional hybrid protein may have an amino acid sequence having at least about 85%, 90%, 95%, 96%, 97%, 98%, or 99% sequence homology to the amino acid sequence of SEQ ID NO:11 (encoded by nucleic acid sequence SEQ ID NO:12).

Most typically, but not necessarily, the extracellular binding domain of the CAR will be a scFv or other natural or synthetic binding portion that specifically binds an antigen of interest. Especially suitable binding portions include small antibody fragments with single, dual, or multiple target specificities, beta barrel domain binders, phage display fusion proteins, etc. Among other suitable extracellular binding domains, preferred domains will specifically bind to a tumor-specific antigen, a tumor associated antigen, or a patient- and tumor-specific antigen. Tumor-specific antigens include, without limitation, NKG2D ligands, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAP, WT-1, PSMA, NY-ESO1, AFP, CEA, CTAG1B, CD33, CD19, CD20, HER-2, CD123, PD-L1, IGF1R, CSPG4, BCMA, and/or B7-H4. Additional non-limiting tumor-specific antigens are described, by way of non-limiting example, in US2013/0189268; WO 1999024566 A1; U.S. Pat. No. 7,098,008; and WO 2000020460, each of which is incorporated herein by reference in its entirety. Likewise, other preferred domains will specifically bind to a (pathogenic) virus-specific antigen, such as an antigen of an HIV virus (e.g., gp120), an HPV virus, an RSV virus, an influenza virus, an ebolavirus, or an HCV virus.

In additionally preferred embodiments, a luminescence labeled modified NK-92 cell includes a nucleic acid encoding a homing receptor operably linked to a promoter. In some aspects, the homing receptor is a G protein-coupled receptor (GPCR), a chemokine receptor, a cytokine receptor, a cell adhesion molecule, a selectin, or an integrin. Typically, the chemokine receptor is CCR7, CXCR2, or the receptor for CXCL14, and the cell adhesion molecule is L-selectin (CD62L), α4β7 integrin, LPAM-1, or LFA-1. In further embodiments, the luminescence labeled modified NK-92 cell includes a nucleic acid encoding the GPCR receptor operably linked to a promoter and also includes a chimeric antigen receptor (CAR) as disclosed herein also operably linked the promoter.

Accordingly, a luminescence labeled modified NK-92 cells as disclosed herein that is genetically engineered to express a homing receptor and/or a chimeric antigen receptor (CAR) allows for the in vivo localization of these cells. This localization capability is a valuable tool for identifying NK-92 cell therapies that target desired anatomical locations (e.g., lungs, kidneys, spleen, liver, brain, and/or bones). These desired anatomical locations may include any organ or tissue in the body harboring cancer cells, most especially metastasized cancer cells which are not localized to a tumor or tumor site. For example, using the luminescence labeled modified NK-92 cells as disclosed herein, cells capable of localizing to the brain and/or bone may be identified.

### Examples

In view of the above, and as provided in more detail below, one exemplary method entailed transforming NK-92 cells with a firefly luciferase gene. An injection dose of luciferase labeled NK-92 (FLuc-aNK or FLuc-NK-92) cells was prepared with 1.0×10⁷ cells after irradiation and resuspended in 200 μl PBS. Where indicated, the control “vehicle control” dose was 200 μl of PBS alone. More specifically:

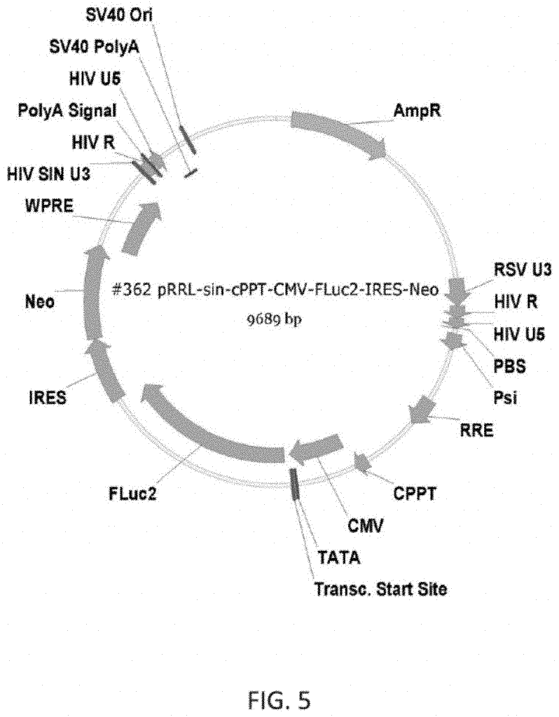

The objective of a first study was to use in vivo bioluminescent imaging as a tool to track the distribution and persistence of Firefly Luciferase (FLuc) labeled aNK cells in a murine host. Here, aNK cells were transfected with a plasmid according to FIG. 5 to so produce FLuc-aNK cells. The FLuc-aNK cells will be cultured in X-Vivo10 medium (Cat #BE02-055Q) supplemented with 5% heat inactivated human AB serum (Cat #IPLA-SERABHI, Innovative Research), 500 IU/mL IL-2 (Cat #CYT-209, Prospec Bio) and 1 mg/mL G418 (Cat #20-234-CI, Corning) following standard NK cell culture protocol. FLuc-aNK cells were irradiated with a dose of 1000 cGy prior to administration.

The objective of a second study was to use in vivo bioluminescent imaging as a tool to track the distribution and persistence of Firefly Luciferase (FLuc) labeled aNK cells after repeated dosing in varied murine hosts. Here, aNK cells were transfected with a plasmid according to FIG. 5 to so produce FLuc-aNK cells. The FLuc-aNK cells will be cultured in X-Vivo10 medium (Cat #BE02-055Q) supplemented with 5% heat inactivated human AB serum (Cat #IPLA-SERABHI, Innovative Research), 500 IU/mL IL-2 (Cat #CYT-209, Prospec Bio) and 1 mg/mL G418 (Cat #20-234-CI, Corning) following standard NK cell culture protocol. FLuc-aNK cells were irradiated with a dose of 1000 cGy prior to administration.

For all strains of animals, dosing started at 1 day after randomization and was repeated at a frequency of twice a week for 4 consecutive weeks. All doses were administered intravenously via the lateral tail vein. The 3 animals in Group A received 200 μL PBS, while the 3 animals in Group B received 1×10⁷ irradiated FLuc-aNK cells in 200 μL PBS.

For all animals, 1 day after randomization, 3 mice in Group A received a single subcutaneous dose of 50 μL PBS on the flank. The 12 (or 3 Balb/c) animals in Group B received a single subcutaneous injection of 2.5×10⁶ irradiated FLuc-aNK cells in 50 μL PBS on the flank, while the 12 (or 3 Balb/c) animals in Group C received a single intravenous dose of 1×10⁷ irradiated FLuc-aNK cells in 200 μL PBS via the lateral tail vein.

Imaging: Animals were imaged in vivo for the expression of FLuc as an indicator of aNK cell distribution. Imaging time points were: 2, 24, 48 and either 72 hours or 1 week (depending on the 48 h imaging result) post NK cell inoculation for Group B animals (N=3); and 2, 24, 48 and 72 hours for Group C animals (N=3). PBS injected mice were imaged at 2 hours only (N=3). Immunocompromised mice were imaged only once at the respective time points whereas immunocompetent Balb/c mice were imaged repeatedly.

Preparation of D-Luciferin: Thaw D-Luciferin (Potassium Salt GoldBio Catalog #LUCK; Sodium Salt GoldBio Catalog #LUCNA) at room temperature and dissolve in PBS (no calcium or magnesium) to a final concentration of 15 mg/mL. Sterilize the Luciferin solution by going through a 0.22 μm filter pre-wetted with sterile H₂O.

D-Luciferin administration: The animals were administered 10 μL per gram body weight of D-Luciferin solution intraperitoneally, followed by 15 minutes incubation for maximum luciferase signal to plateau.

Imaging procedure: Animals were anesthetized by inhalation of 2-5% isoflurane with oxygen and placed in a heated imaging chamber in the supine position and acquired for 5 seconds-5 minutes depending on the signal intensity. Animals with fur were shaved in relevant areas prior to imaging. Tissues were collected after euthanasia immediately following imaging.

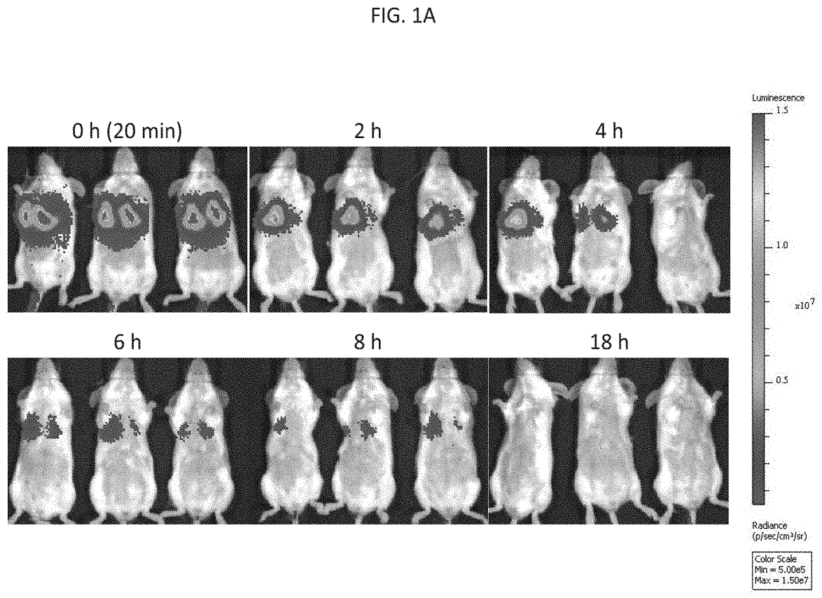

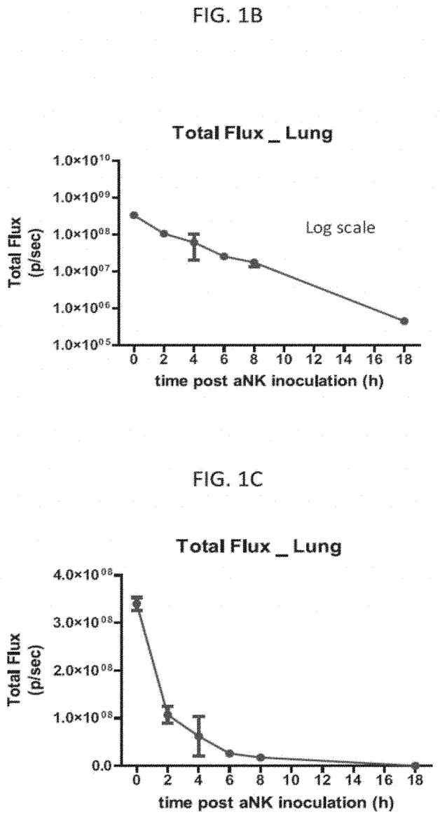

With reference to FIG. 1A, 18 SCID (severe combined immunodeficient) female mice (Taconic Biosciences) were intravenously injected through the lateral tail vein with a 200 μl dose of the luciferase labeled NK-92 cells. Luminescence in vivo imaging of a set of three of the injected mice was carried out immediately (e.g., time at 0 hours, approximately 20 minutes after injection), and at 2 hours, 4 hours, 6 hours, 8 hours, and 18 hours, as indicated. The injected luciferase-labeled NK-92 cells predominantly homed to the lungs and the luminescence signal decreased over time with the most significant decrease occurring in the early time points. The total luminescence signal of the 3 mice at each time point was measured as the luminous energy per unit time (p/sec) and quantified on a log scale as shown in FIG. 1B and a linear scale as shown in FIG. 1C.

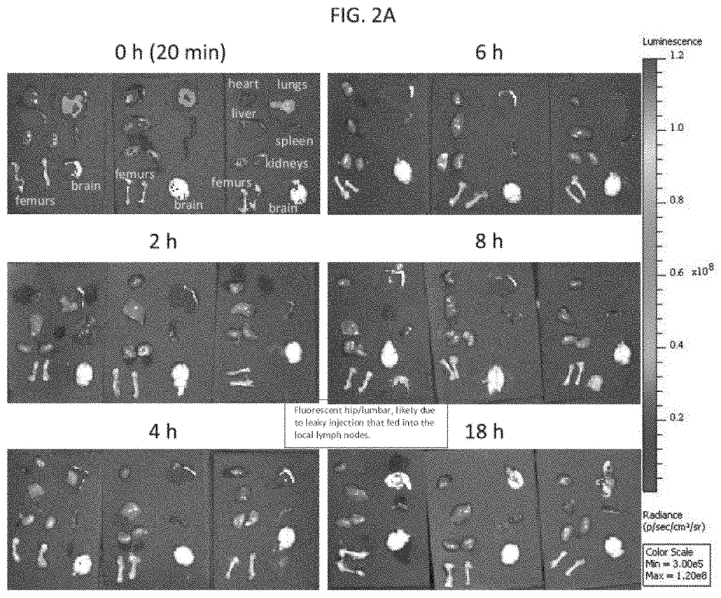

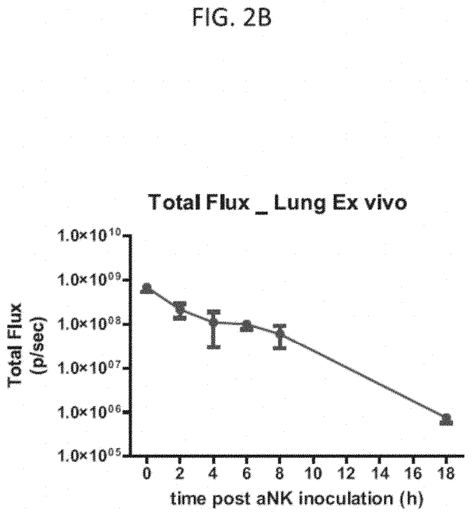

With reference to FIG. 2A, after each imaging time point, the injected SCID mice shown in FIG. 1A were sacrificed and each of the heart, lungs, liver, spleen, kidneys, femurs, and brain were extracted and imaged ex vivo. At the initial 0 hour time point, all tested tissues showed a low level of luminescence, possibly due to the distribution of the labeled NK-92 cells in the blood. The lungs were shown to be the main organ to which the labeled NK-92 localized after injection. Low levels of signal were detected in the spleen in some animals at 6 and 8 hours. In general, the luminescence signal decreased rapidly after the initial 0 hour time point. The total luminescence signal of the collected organs of the mice at each time point was measured as the luminous energy per unit time (p/sec) and quantified on a log scale as shown in FIG. 2B.

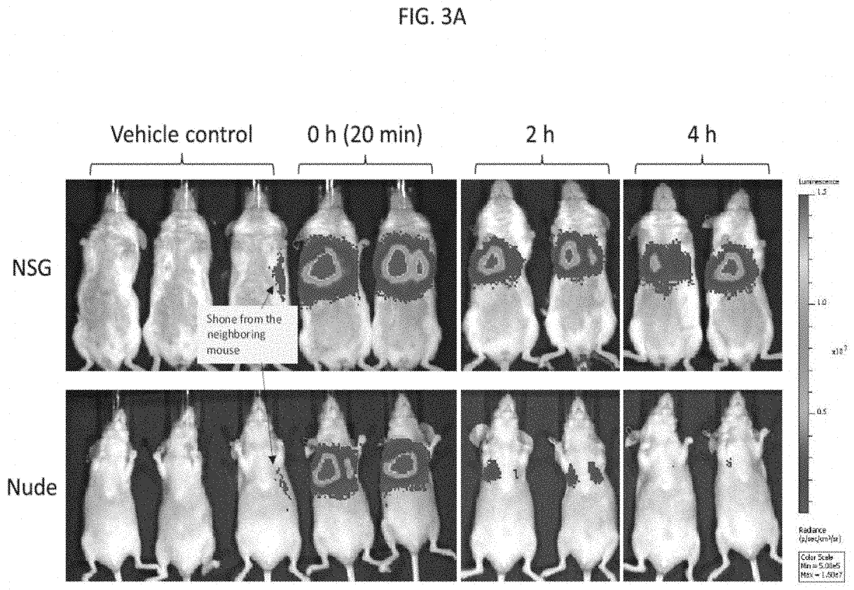

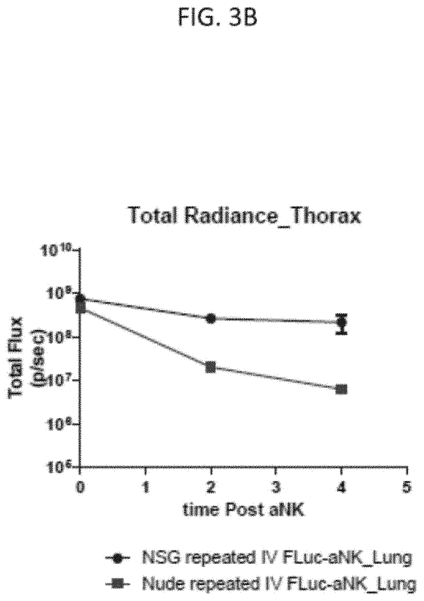

With reference to FIG. 3A, 9 NSG (Non-obese diabetic (NOD)-scid-gamma) female mice (The Jackson Laboratory) and 9 nude female mice (The Jackson Laboratory) were intravenously injected through the lateral tail vein with a 200 μl dose of PBS alone (“vehicle control”) or a 200 μl dose of the luciferase labeled NK-92 irradiated cells twice a week for 4 weeks (8 total doses/mouse) with images taken at the indicated time point after the last dose. Luminescence in vivo imaging of a set of three each of the control injected NSG and the nude mice was carried out immediately (e.g., time at 0 hours, approximately 20 minutes after injection), followed by luminescence in vivo imaging of a set of two each of the NSG mice and nude mice injected with the luciferase labeled NK-92 cells at 2 hours and 4 hours, as indicated. In general, the luminescence signal indicated the luciferase labeled NK-92 cells homed to (localized to) the lungs or thorax region of both the mice strains. Notably, the luminescence signal decreased over time and more drastically in the nude mice than in the NSG mice. The total luminescence signal of the NSG or nude mice at each time point was measured as the luminous energy per unit time (p/sec) and quantified on a log scale as shown in FIG. 3B.

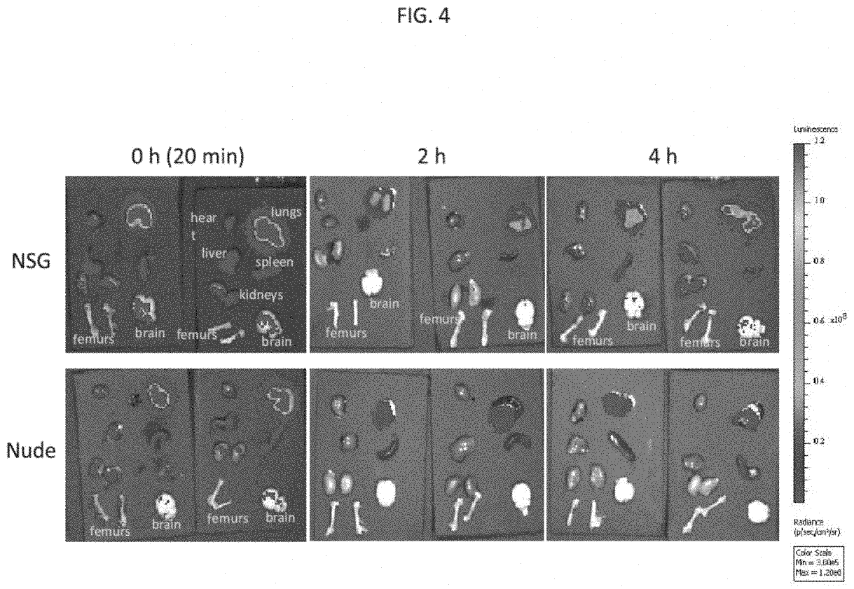

With reference to FIG. 4, after each imaging time point, the injected NSG or nude mice shown in FIG. 3A were sacrificed and each of the heart, lungs, liver, spleen, kidneys, femurs, and brain were extracted and imaged ex vivo. At the initial 0 hour time point, all tested tissues showed a low level of luminescence, possibly due to the distribution of the labeled NK-92 cells in the blood. The lungs were shown to be the main organ to which the labeled NK-92 localized after injection from 0 to 4 hours post injection. Without being bound by any particular theory, inventors note that the discrepancies between the in vivo images in FIG. 3A and the corresponding ex vivo images shown in FIG. 4 may be artifacts of the ex vivo imaging and/or caused by the fact that the luciferase expression requires oxygen (O₂) and ATP which are no longer supplied when the animal is sacrificed.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of assaying in vivo localization of luminescence labeled activated natural killer (aNK) cells in a subject, comprising:
administering a dose of luminescence labeled aNK cells to the subject; and
imaging the subject with bioluminescence imaging (BLI).

2. The method of claim 1, wherein the luminescence labeled aNK cells is transfected with a recombinant nucleic acid expressing a luciferase protein.

3. The method of claim 1, wherein the luminescence labeled aNK cells are modified NK-92 cells.

4. The method of claim 3, wherein the modified NK-92 cells are selected from the group consisting of NK-92-CD16, NK-92-CD16-γ, NK-92-CD16-ζ, NK-92-CD16(F176V), NK-92MI, and NK-92CI.

5. The method of claim 1, wherein the luminescence labeled aNK cells are modified NK-92 cells transfected with a membrane bound recombinantly expressed chimeric antigen receptor (CAR) that comprises in a single polypeptide chain an extracellular binding domain, a hinge domain, a transmembrane domain, and a FcεRIγ signaling domain.

6. The method of claim 5, wherein the extracellular binding domain specifically binds to a tumor-specific antigen, a tumor associated antigen, or a patient- and tumor-specific antigen, or a virus-specific antigen.

7. The method of claim 1, wherein the dose of luminescence labeled aNK cells comprises 1.0×10⁶ to 1.0×10⁹ cells.

8. A method of identifying a luminescence labeled activated natural killer (aNK) cells capable of targeting a selected anatomical location in a subject, the method comprising:
administering a dose of luminescence labeled aNK cells to the subject; and
imaging the subject with bioluminescence imaging (BLI) at the selected anatomical location.

9. The method of claim 8, wherein the luminescence labeled aNK cells is transfected with a recombinant nucleic acid expressing a luciferase protein.

10. The method of claim 8, wherein the selected anatomical location is selected from lungs, heart, spleen, kidneys, liver, bone, and/or brain.

11. The method of claim 8, wherein the luminescence labeled aNK cells are modified NK-92 cells.

12. The method of claim 11, wherein the modified NK-92 cells are selected from the group consisting of NK-92-CD16, NK-92-CD16-γ, NK-92-CD16-ζ, NK-92-CD16(F176V), NK-92MI, or NK-92CI.

13. The method of claim 8, wherein the luminescence labeled aNK cells are modified NK-92 cells transfected with a membrane bound recombinantly expressed chimeric antigen receptor (CAR) that comprises in a single polypeptide chain an extracellular binding domain, a hinge domain, a transmembrane domain, and a FcεRIγ signaling domain.

14. The method of claim 8, wherein the dose of luminescence labeled aNK cells comprises 1.0×10⁶ to 1.0×10⁹ cells.

15. A composition for in vivo bioluminescence imaging, comprising
a modified NK-92 cell genetically modified to produce luminescence, and to recombinantly express an Fc receptor, a chimeric antigen receptor (CAR), a cytokine, or a homing receptor.

16. The composition of claim 15, wherein the NK-92 cell is genetically modified to recombinantly express a luciferase protein.

17. The composition of claim 15, wherein the modified NK-92 cells are selected from the group consisting of NK-92-CD16, NK-92-CD16-γ, NK-92-CD16-ζ, NK-92-CD16(F176V), NK-92MI, or NK-92CI.

18. The composition of claim 15, wherein the modified NK-92 cells are transfected with a membrane bound recombinantly expressed chimeric antigen receptor (CAR) that comprises in a single polypeptide chain an extracellular binding domain, a hinge domain, a transmembrane domain, and a FcεRIγ signaling domain.

19. The composition of claim 18, wherein the extracellular binding domain comprises a scFv.

20. The composition of claim 18, wherein the extracellular binding domain specifically binds to a tumor-specific antigen, a tumor associated antigen, a patient- and tumor-specific antigen, or a virus-specific antigen.


SEQUENCE LISTING (12 sequences)

SEQ ID NO: 1 - protein, 254 aa, Homo sapiens
  MISC_FEATURE (1)..(254): HUMAN Low affinity immunoglobulin gamma Fc region receptor III-A
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 2 - protein, 254 aa, Homo sapiens
  MISC_FEATURE (1)..(254)
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 3 - DNA, 765 bp, Homo sapiens
  misc_feature (1)..(765): High Affinity Variant Immunoglobulin Gamma Fc Region Receptor III-A nucleic acid sequence (full length form)
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 4 - protein, 41 aa, Homo sapiens
  MISC_FEATURE (1)..(41): FceRIg intracellular (cytoplasmic) domain
LKIQVRKAAITSYEKSDGVYTGLSTRNQETYETLKHEKPPQ

SEQ ID NO: 5 - protein, 113 aa, Homo sapiens
  MISC_FEATURE (1)..(113): CD3z signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 28 aa, Homo sapiens
  MISC_FEATURE (1)..(28): CD28 Transmembrane domain
FWVLVVVGGVLACYSLLVTVAFIIFWVR

SEQ ID NO: 7 - DNA, 84 bp, Homo sapiens
  misc_feature (1)..(84): CD28 for the Transmembrane Region Only (Minus ITAM or Intracellular Sequence)
ttttgggtgctggtggtcgtgggcggagtgctggcttgttattctctgctggtcaccgtggccttcatcatcttttgggtccga

SEQ ID NO: 8 - protein, 54 aa, Homo sapiens
  MISC_FEATURE (1)..(54): CD8 Hinge Region
FVPVFLPAKPTTTPAPRPPTPAPTIASQPLSLRPEACRPAAGGAVHTRGLDFAC

SEQ ID NO: 9 - protein, 64 aa, Homo sapiens
  MISC_FEATURE (1)..(64): CD8 Hinge Region
LSNSIMYFSHFVPVFLPAKPTTTPAPRPPTPAPTIASQPLSLRPEACRPAAGGAVHTRGLDFAC

SEQ ID NO: 10 - DNA, 192 bp, Homo sapiens
  misc_feature (1)..(192): CD8a for the Hinge Region
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgc

SEQ ID NO: 11 - protein, 133 aa, Artificial Sequence
  Hybrid comprising CD8 hinge region, CD28 transmembrane, and FceRIgamma signaling domain amino acid sequence
LSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 12 - DNA, 399 bp, Artificial Sequence
  Hybrid comprising CD8 hinge region, CD28 transmembrane, and FceRIgamma signaling domain
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag